(19) United States
(12) Patent Application Publication
(10) Pub. No.: US 2026/0250357 A1
(43) Pub. Date: Aug. 27, 2026

(54) FUSION PROTEIN COMPRISING TYPE I COLLAGEN AND ELASTIN, AND USE THE SAME

(71) Applicant: Nature Intro Pharm Co., Ltd., Incheon (KR)

(72) Inventors: Kyoung Hyoun LEE, Gunpo-si (KR); Tae Won CHOI, Bucheon-si (KR); Eun Young LEE, Pohang-si (KR)

(21) Appl. No.: 19/549,578

(22) Filed: Feb. 25, 2026

(30) Foreign Application Priority Data
Feb. 25, 2025 (KR) 10-2025-0024338

(51) Int. Cl. C07K 14/78; A61K 8/65; A61Q 19/08; C12N 15/63

(52) U.S. Cl. CPC C07K 14/78; A61K 8/65; A61Q 19/08; C12N 15/63; C07K 2319/00

(57) ABSTRACT

The present invention relates to a fusion protein comprising type I collagen and elastin, and to a use thereof. Specifically, the fusion protein of type I collagen and elastin according to the present invention promotes collagen synthesis and inhibits collagenase activity without showing cytotoxicity to cells, and thus can be usefully used as a functional material for skin improvement.

### CROSS-REFERENCE TO RELATED APPLICATION

This application claims priority to Korean Patent Application No. KR 10-2025-0024338 filed Feb. 25, 2025, the entire contents of which are incorporated herein by reference.

### REFERENCE TO AN ELECTRONIC SEQUENCE LISTING

The instant application contains a Sequence Listing in xml (ST.26) format (File Name: FP25-0220-US_seq.xml; Size: 13,743 bytes; Date of Creation: Jan. 8, 2026) and incorporated herein by reference in its entirety.

### TECHNICAL FIELD

The present invention relates to a fusion protein comprising type I collagen and elastin, and use the same.

### BACKGROUND ART

The skin is largely composed of three layers: the epidermis, dermis, and subcutaneous adipose tissue, and functions to protect the body from the external environment. Among these layers, the epidermis is classified, in order, into the stratum corneum, granular layer, spinous layer, and basal layer, and the stratum corneum, the outermost layer, is formed in a brick and mortar structure. Keratinocytes act as bricks, and the lipids between the cells serve as mortar that firmly binds the keratinocytes, thereby forming a skin barrier that suppresses evaporation of moisture to the outside and protects the body against external physical and chemical stimuli.

Additionally, the stratum corneum contains hydrophilic natural moisturizing factors that retain moisture and contribute to skin hydration, and an appropriate oil formed by sebum and sweat secreted from the sebaceous and sweat glands minimizes water loss from the skin. However, even healthy skin may become dry due to various factors such as stress, ultraviolet (UV) radiation, aging, or environmental pollution.

Additionally, the skin prevents the ingress of microorganisms from outside and plays an important role in maintaining bodily moisture and temperature. This function is performed primarily by the epidermis, which comprises an extracellular matrix associated with skin elasticity and flexibility. Skin aging can be classified into physiological aging and photoaging caused by ultraviolet (UV) radiation. Dermal aging associated with skin aging results from reduced numbers of fibroblasts and a diminished capacity to synthesize proteins that maintain the dermal layer.

In this regard, Korean Patent Publication No. 10-2021-0056984 discloses that an extract of Panax notoginseng can be used for skin improvement—including skin regeneration, anti-inflammation, improvement of atopic conditions, and moisturizing—through promoting the proliferation of skin stem cells, inhibiting NO production, and enhancing activation of PPAR-α.

### SUMMARY OF THE INVENTION

### Technical Problem

An object of the present invention is to provide a fusion protein of type I collagen and elastin, a polynucleotide encoding the fusion protein, an expression vector comprising the polynucleotide, and a transformant transformed with the expression vector.

Another object of the present invention is to provide a method for preparing the fusion protein according to the present invention using the transformant.

Still another object of the present invention is to provide a use for the fusion protein, polynucleotide, expression vector, or transformant.

### Solution to the Problem

To achieve the above objects, the present invention provides a fusion protein of type I collagen and elastin.

In addition, the present invention provides a polynucleotide encoding the fusion protein.

In addition, the present invention provides an expression vector comprising the polynucleotide.

In addition, the present invention provides a transformant transformed with the expression vector.

In addition, the present invention provides a method for preparing a fusion protein of collagen and elastin according to the present invention, comprising the step of culturing the transformant.

In addition, the present invention provides a cosmetic composition for skin improvement comprising the fusion protein, expression vector, or transformant as an active ingredient.

Furthermore, the present invention provides a health functional food for skin improvement comprising the fusion protein, expression vector, or transformant as an active ingredient.

### Advantageous Effects of the Invention

The fusion protein of type I collagen and elastin according to the present invention promotes collagen synthesis and inhibits collagenase activity without showing cytotoxicity to cells, and thus can be usefully used as a functional material for skin improvement.

### BRIEF DESCRIPTION OF DRAWINGS

FIG. 1 is a schematic diagram of an expression vector expressing a fusion protein of type I collagen al and elastin, prepared in an embodiment of the present invention.

FIG. 2 is an illustration showing the results of confirming the expression of the fusion protein of type I collagen al and elastin in an embodiment of the present invention (M: size marker, 1: No IPTG added, 2: Whole fraction with IPTG added, 3: Insoluble fraction with IPTG added, 4: Soluble fraction with IPTG added).

FIG. 3 is an illustration showing the results of confirming the purified fusion protein in an embodiment of the present invention.

FIG. 4 is a graph showing the results of confirming the cell proliferation-promoting effect of the fusion protein in an embodiment of the present invention.

FIG. 5 is a graph showing the results of confirming the collagen synthesis-promoting effect of the fusion protein in an embodiment of the present invention.

FIG. 6 is a graph showing the results of confirming the collagenase activity-inhibiting effect of the fusion protein in cells in an embodiment of the present invention.

FIG. 7 is a graph showing the results of confirming the collagenase activity-inhibiting effect of the fusion protein by substrate-enzyme reaction in an embodiment of the present invention.

### DETAILED DESCRIPTION OF THE INVENTION

Hereinafter, the present invention will be described in detail.

The present invention provides a fusion protein of type I collagen and elastin.

The term “collagen” as used herein is a fibrous protein found mainly in most animals, especially mammals, and accounts for most of all connective tissues in the body, such as skin and cartilage. Collagen has a rope-like form in which three polypeptide molecules are twisted in a triple helix, and each polypeptide chain has glycine and proline arranged alternately, with other types of amino acids inserted in between. The collagen is classified into type I to VIII, among which type IV, type VI, type VII, and type VIII collagens are non-fibrillar collagens. Type I collagen is a fibrillar collagen that is most abundantly present in the body, and is mainly distributed in bones and dermis.

The type I collagen may be type I collagen α1, and the type I collagen al may comprise any type of type I collagen α1 that is known in the art. For example, the amino acid sequence of the type I collagen α1 is well known in the art, and may also comprise variants in which some amino acids are substituted, deleted, or inserted in the amino acid sequence while maintaining the activity of collagen. More specifically, the type I collagen α1 may be a polypeptide consisting of the amino acid sequence set forth in SEQ ID NO: 7. In addition, the polypeptide may comprise one having 90% or more, 93% or more, 95% or more, 96% or more, 97% or more, 98% or more, or 99% or more homology with the amino acid sequence set forth in SEQ ID NO: 7.

In one aspect of the present invention, the type I collagen α1 may be type I collagen α1 or a fragment thereof. The fragment of type I collagen α1 may be a polypeptide consisting of the 464th to 626th amino acids from the N-terminus of the amino acid sequence constituting type I collagen α1. Specifically, the fragment of type I collagen α1 may be a polypeptide consisting of the amino acid sequence set forth in SEQ ID NO: 1. The type I collagen α1 or a fragment thereof may also comprise variants in which some amino acids are substituted, deleted, or inserted, as long as the activity of type I collagen α1 is maintained. In addition, the type I collagen α1 or a fragment thereof may also comprise a polynucleotide encoding it. The polynucleotide is obvious to those skilled person in the art as long as the sequence of the protein is known. For example, the polynucleotide may consist of the base sequence set forth in SEQ ID NO: 2. The polynucleotide may comprise variants in which some bases constituting it are substituted, deleted, or inserted. In addition, the polynucleotide may comprise one having 90% or more, 93% or more, 95% or more, 96% or more, 97% or more, 98% or more, or 99% or more homology with the base sequence set forth in SEQ ID NO: 2.

The term “elastin” as used herein is a protein that exists in connective tissue and has elasticity, and is involved in the flexibility and elasticity of tissues. Elastin is an insoluble protein and has strong chemical resistance to acids, bases, and proteolytic enzymes, and resistance to heat, but is decomposed by pepsin and trypsin.

The elastin may comprise any type of elastin that is known in the art. For example, the amino acid sequence of elastin is well known in the art, and may also comprise variants in which some amino acids are substituted, deleted, or inserted in the amino acid sequence while maintaining the activity of elastin. More specifically, the elastin may be a polypeptide consisting of the amino acid sequence set forth in SEQ ID NO: 8. In addition, the polypeptide may comprise one having 90% or more, 93% or more, 95% or more, 96% or more, 97% or more, 98% or more, or 99% or more homology with the amino acid sequence set forth in SEQ ID NO: 8.

In another aspect of the present invention, the elastin may be elastin or a fragment thereof. Specifically, the fragment of elastin may be a polypeptide consisting of the 421st to 543rd amino acids from the N-terminus of the amino acid sequence constituting elastin. Specifically, the fragment of elastin may be a polypeptide consisting of the amino acid sequence set forth in SEQ ID NO: 3. The elastin or a fragment thereof may also comprise variants in which some amino acids are substituted, deleted, or inserted, as long as the activity of elastin is maintained. In addition, the elastin or a fragment thereof may also comprise a polynucleotide encoding it. The polynucleotide is obvious to those skilled person in the art as long as the sequence of the protein is known. For example, the polynucleotide may consist of the base sequence set forth in SEQ ID NO: 4. The polynucleotide may comprise variants in which some bases constituting it are substituted, deleted, or inserted. In addition, the polynucleotide may comprise one having 90% or more, 93% or more, 95% or more, 96% or more, 97% or more, 98% or more, or 99% or more homology with the base sequence set forth in SEQ ID NO: 4.

In one embodiment of the present invention, the fusion protein may be a polypeptide consisting of the amino acid sequence set forth in SEQ ID NO: 5. In addition, the polypeptide may also comprise a polynucleotide encoding it. The polynucleotide is obvious to those skilled person in the art as long as the sequence of the protein is known. For example, the polynucleotide may consist of the base sequence set forth in SEQ ID NO: 6. The polynucleotide may comprise variants in which some bases constituting it are substituted, deleted, or inserted. In addition, the polynucleotide may comprise one having 90% or more, 93% or more, 95% or more, 96% or more, 97% or more, 98% or more, or 99% or more homology with the base sequence set forth in SEQ ID NO: 6.

The protein or polypeptide described above may comprise conservative substitutions of some amino acids constituting it. The term “conservative substitution” means that one amino acid is substituted with another amino acid having similar structural or chemical properties. For example, the type I collagen or a fragment thereof, elastin or a fragment thereof, and fusion protein according to the present invention may have one or more conservative substitutions of amino acids while still retaining biological activity. Such amino acid substitutions can generally occur based on similarities in polarity, charge, solubility, hydrophobicity, hydrophilicity, and amphipathicity of the residues. Specifically, positively charged amino acids may be arginine, lysine, or histidine; negatively charged amino acids may be glutamic acid or aspartic acid; aromatic amino acids may be phenylalanine, tryptophan, or tyrosine; and hydrophobic amino acids may be alanine, valine, isoleucine, leucine, methionine, phenylalanine, tyrosine, or tryptophan. In addition, amino acids can be classified into amino acids having electrically charged side chains and amino acids having uncharged side chains. Amino acids with charged side chains comprise aspartic acid, glutamic acid, lysine, arginine, and histidine, and amino acids with uncharged side chains can be further classified into nonpolar amino acids or polar amino acids. Nonpolar amino acids may be glycine, alanine, valine, leucine, isoleucine, methionine, or proline, and polar amino acids may be serine, threonine, cysteine, asparagine, or glutamine. The conservative substitution is expected to exhibit the same or similar activity even if some amino acids constituting the protein or polypeptide are substituted.

In addition, the present invention provides a polynucleotide encoding the fusion protein.

The fusion protein encoded by the polynucleotide according to the present invention may have the characteristics as described above.

The polynucleotide may comprise DNA, RNA, or modified forms thereof. For example, the polynucleotide may be a cDNA, an mRNA, or a modified forms thereof. When the polynucleotide is DNA or modified DNA, it may comprise adenine (A), thymine (T), guanine (G), cytosine (C), or modified forms thereof. Meanwhile, when the polynucleotide is RNA or modified RNA, it may comprise adenine, uridine (U), guanine, cytosine, or modified forms thereof. Specifically, the modified DNA or RNA may be one or more selected from the group consisting of 5-methylcytidine (5mC), N6-methyladenosine (m6A), 3,2′-O-dimethyluridine (m4U), 2-thiouridine (s2U), 2′-fluorouridine, pseudouridine, 2′-O-methyluridine (Um), 2′-deoxyuridine (2′dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), N2,7-dimethylguanosine (m-2,7G), N2,N2,7-trimethylguanosine (m-2,2,7G), and N1-methyl-pseudouridine.

Therefore, when the polynucleotide is in the form of RNA, tyrosine in the base sequence constituting the polynucleotide encoding the fusion protein herein may be expressed as substituted with uridine, and when the polynucleotide is in the form of modified DNA or modified RNA, adenine, thymine, guanine, cytosine, and uridine in the base sequence constituting the polynucleotide encoding the fusion protein herein may be expressed as substituted with their modified forms, respectively.

Due to the degeneracy of the codon, the polynucleotide may comprise various modifications in the coding region within a range that does not change the amino acid sequence of the protein expressed from the coding region, taking into account the codons preferred in the organism used to express the fusion protein, in the process of expressing the fusion protein therefrom. Therefore, the polynucleotide may comprise any sequence as long as it is a nucleotide sequence encoding the fusion protein according to the present invention.

For example, the polynucleotide may consist of the base sequence set forth in SEQ ID NO: 6. The polynucleotide may comprise variants in which some bases constituting it are substituted, deleted, or inserted. In addition, the polynucleotide may comprise one having 90% or more, 93% or more, 95% or more, 96% or more, 97% or more, 98% or more, or 99% or more homology with the base sequence set forth in SEQ ID NO: 6.

In addition, the present invention provides an expression vector comprising the polynucleotide.

The polynucleotide comprised in the expression vector may have the characteristics as described above.

The term “expression vector” as used herein refers to a vector capable of expressing a target foreign gene in a host cell, meaning a vector that comprises essential regulatory elements operably linked to allow the expression of a gene insert. The term “operably linked” means that a nucleic acid expression control sequence and a nucleic acid sequence encoding a target protein are functionally linked to perform a general function. Operable linkage with a recombinant vector can be prepared by methods well known in the ordinary technical field, and site-specific DNA cleavage and ligation can also be performed using enzymes well known in the art.

The expression vector may be one in which a polynucleotide encoding a desired target protein is introduced into a conventionally known expression vector. The preparation of the expression vector is well known in the art, and various vectors known to be capable of expressing the target protein can be used as the vector for preparing the expression vector. For example, the expression vector may comprise plasmid vectors, cosmid vectors, bacteriophage vectors, and viral vectors.

The expression vector may comprise signal sequences for membrane targeting or secretion, in addition to expression control elements such as a promoter, an initiation codon, a termination codon, a polyadenylation signal, and an enhancer. The initiation codon and termination codon are generally considered part of the nucleotide sequence encoding the target protein, must function in the individual when the gene construct is administered, and must be in frame with the coding sequence. The promoter may be a constitutive or inducible promoter, and the promoter may be selected according to the type of transformant into which the expression vector will be transformed. For example, when the transformant is a prokaryotic cell, the promoter may be a lac, tac, T3, or T7 promoter. When the transformant is a eukaryotic cell, the promoter may be selected from promoters derived from viruses including simian virus 40 (SV40), mouse mammary tumor virus (MMTV), the long terminal repeat (LTR) of human immunodeficiency virus (HIV), Moloney virus, cytomegalovirus (CMV), Epstein-Barr virus (EBV), and Rous sarcoma virus (RSV), or from cellular genes including β-actin, human hemoglobin, human muscle creatine, and human metallothionein.

The expression vector may comprise a selectable marker for selecting transformants containing the vector. As the selectable marker, markers that confer a selectable phenotype such as drug resistance, auxotrophy, resistance to cytotoxic agents, or expression of surface proteins may be used. In an environment treated with a selective agent, only cells expressing the selectable marker survive, so transformed cells can be selected. In addition, if the vector is a replicable expression vector, it may comprise an origin of replication, which is a specific nucleic acid sequence where replication is initiated.

The expression vector may be one used in the art or a commercially available product. For example, the expression vector may be selected from: pET21a, pET, pRSET, pBluescript, pGEX-2T, and pUC series vectors; bacterial plasmids derived from Escherichia coli, such as ColE1, pCR1, pBR322, pMB9, and derivatives thereof; plasmids having a broader host range, such as RP4; phage DNA exemplified by bacteriophage lambda derivatives including λgt10, λgt11, and MM989; and plasmids derived from other DNA phages, such as M13 and other filamentous single-stranded DNA phages.

In addition, the present invention provides a transformant transformed with the expression vector.

The term “transformant” as used herein means that the genetic traits have been changed by including foreign genetic material. The transformant according to the present invention is easy to manipulate genetically, can be applied to various expression systems, and can be used for mass culture. The transformant may be a eukaryotic cell derived from yeast, fungi, protozoa, plants, higher plants, amphibians, or mammals, or a prokaryotic cell such as Escherichia coli.

The transformation may be performed according to methods known in the art, and may be appropriately modified by those skilled person in the art according to the type and characteristics of the host to be transformed. For example, the transformation may comprise the transformation method using glass beads known by Kindle (1990), the transformation method of protoplasts using the calcium/polyethylene glycol method, electroporation, microinjection, particle bombardment, electropolation, the Agrobacterium-mediated method, the gene gun method, or physical introduction methods.

In addition, the present invention provides a method for preparing a fusion protein of collagen and elastin according to the present invention, comprising the step of culturing the transformant.

The transformant used in the preparation method according to the present invention may have the characteristics as described above.

The culturing may be performed by methods well known in the art, and may also be performed by methods appropriately modified by those skilled person in the art as needed. Specifically, the culturing may be performed by inoculating the strain into a culture medium. At this time, the culture medium may comprise a carbon source, a nitrogen source, and inorganic salts. The carbon source, nitrogen source, and inorganic salts are well known in the art, and may be appropriately selected and used by those skilled person in the art as needed. For example, the carbon source may be sugars and carbohydrates such as glucose, sucrose, lactose, fructose, maltose, starch, cellulose; oils such as soybean oil, sunflower oil, castor oil, coconut oil; and fats, fatty acids such as palmitic acid, stearic acid, linoleic acid, alcohols such as glycerol, ethanol; and organic acids such as gluconic acid, acetic acid, pyruvic acid. Meanwhile, the nitrogen source may comprise peptone, yeast extract, meat broth, malt extract, corn steep liquor, soybean meal, and urea or inorganic compounds (e.g., ammonium sulfate, ammonium chloride, ammonium phosphate, ammonium carbonate, and ammonium nitrate). In addition, the inorganic salts may comprise phosphorus (P), sodium (Na), potassium (K), magnesium (Mg), sulfur(S), iron (Fe), and chlorine (Cl).

The culturing may be performed under conditions suitable for culturing the transformant.

In addition, the present invention provides a cosmetic composition for skin improvement comprising the fusion protein, expression vector, or transformant as an active ingredient.

The fusion protein, expression vector, or transformant included in the cosmetic composition according to the present invention may have the characteristics as described above.

Meanwhile, the skin improvement may be due to skin wrinkle improvement.

The cosmetic composition of the present invention may comprise the fusion protein, expression vector, or transformant according to the present invention in an amount of 0.1 to 50% by weight, specifically 1 to 10% by weight. The cosmetic composition may be directly applied to the skin for the purpose of skin improvement.

The cosmetic composition may be formulated into commonly prepared cosmetic formulations. The cosmetic composition may be formulated as a solution, suspension, emulsion, paste, gel, lotion, cream, powder, soap, surfactant-containing cleansing, oil, powder foundation, emulsion foundation, wax foundation, and spray. Specifically, it may be a softening toner, nourishing toner, nourishing cream, massage cream, essence, eye cream, cleansing cream, cleansing foam, cleansing water, pack, spray, or powder.

When the formulation of the cosmetic composition of the present invention is a paste, cream, or gel, it may comprise animal oil, vegetable oil, wax, paraffin, starch, tragacanth, cellulose derivatives, polyethylene glycol, silicone, bentonite, silica, talc, zinc oxide, or a mixture thereof as a carrier. In addition, when the formulation of the cosmetic composition is a powder or spray, it may comprise lactose, talc, silica, aluminum hydroxide, calcium silicate, polyamide powder, or a mixture thereof. In particular, in the case of a spray, it may further comprise chlorofluorohydrocarbons, propane/butane, or dimethyl ether.

When the formulation of the cosmetic composition of the present invention is a solution or emulsion, it may comprise a solvent, a solubilizer, an emulsifier, or a mixture thereof as a carrier. Examples thereof include water, ethanol, isopropanol, ethyl carbonate, ethyl acetate, benzyl alcohol, benzyl benzoate, propylene glycol, 1,3-butyl glycol oil, glycerol aliphatic esters, polyethylene glycol, sorbitan fatty acid esters, and the like.

When the formulation of the cosmetic composition of the present invention is a suspension, it may comprise a liquid diluent such as water, ethanol, or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, tragacanth, or a mixture thereof as a carrier. In addition, when the formulation of the cosmetic composition is a surfactant-containing cleansing, it may comprise, as a carrier, an aliphatic alcohol sulfate, an aliphatic alcohol ether sulfate, a sulfosuccinic acid monoester, an isethionate, an imidazolinium derivative, a methyl taurate, a sarcosinate, a fatty acid amide ether sulfate, an alkylamidobetaine, a fatty alcohol, a fatty acid glyceride, a fatty acid diethanolamide, a vegetable oil, a lanolin derivative, an ethoxylated glycerol fatty acid ester, or a mixture thereof.

The cosmetic composition of the present invention may comprise, in addition to the carrier component, antioxidants, stabilizers, solubilizers, moisturizers, pigments, fragrances, UV blockers, colorants, surfactants, or mixtures thereof as an auxiliary agent. As the auxiliary agent, any substance commonly used in the preparation of cosmetic compositions may be used.

Furthermore, the present invention provides a health functional food for skin improvement comprising the fusion protein, expression vector, or transformant as an active ingredient.

The fusion protein, expression vector, or transformant included in the health functional food according to the present invention may have the characteristics as described above.

Meanwhile, the skin improvement may be due to skin wrinkle improvement.

The fusion protein, expression vector, or transformant according to the present invention included as an active ingredient in the health functional food according to the present invention may have the characteristics as described above.

The fusion protein, expression vector, or transformant of the present invention may be added to food as is, or used together with other foods or food ingredients. At this time, the content of the added active ingredient may be determined according to the purpose, and generally may be 0.01 to 90 parts by weight based on the total weight of the health functional food.

The form and type of health functional food are not particularly limited. Specifically, the health functional food may be in the form of tablets, capsules, powders, granules, liquids, and pills. The health functional food may comprise various flavoring agents, sweeteners, or natural carbohydrates as additional ingredients. The sweetener may be a natural or synthetic sweetener, and examples of natural sweeteners include thaumatin, stevia extract, and the like. Meanwhile, examples of synthetic sweeteners include saccharin, aspartame, and the like. In addition, the natural carbohydrates may be monosaccharides, disaccharides, polysaccharides, oligosaccharides, and sugar alcohols.

The health functional food of the present invention may further comprise nutrients, vitamins, electrolytes, flavoring agents, coloring agents, pectic acid and its salts, alginic acid and its salts, organic acids, protective colloid thickeners, pH adjusters, stabilizers, preservatives, glycerin, or alcohol, in addition to the additional ingredients described above. These ingredients may be used independently or in combination. The ratio of the additives may be selected in the range of 0.01 to 0.1 parts by weight per 100 parts by weight of the composition of the present invention.

Hereinafter, the present invention will be described in detail by the following examples, but the following examples are only for illustrating the present invention, and the present invention is not limited by them. Any configuration that has substantially the same configuration as the technical idea described in the claims of the present invention and achieves the same operational effect is comprised in the technical scope of the present invention.

### Example 1. Preparation of a Fusion Protein of Collagen Type I al (COL1A1) and Elastin

A fusion protein of collagen type I al and elastin was prepared as follows.

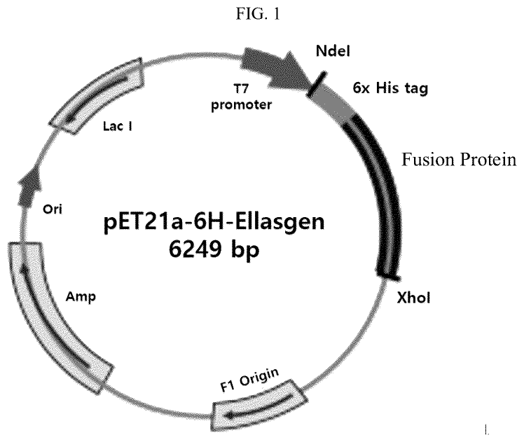

First, a fusion protein DNA (SEQ ID NO: 6) in a form where collagen α1 and elastin were fused was commissioned from Bioneer Corporation and provided in a form cloned into a pBHA vector, which was named pBHA-Ellasgen. Using the expression vector as a template, the fusion protein DNA was amplified by a conventional method using a forward primer (SEQ ID NO: 9: 5′-GGAGATATACATATGCATCACCATCATCACCATGGTCCTCCTGG-3′) including 6× histidine and a reverse primer (SEQ ID NO: 10: 5-GTGGTGGTGCTCGAGTTAGCCAACGCCAGG-3′). The amplified fusion protein DNA was cut with NdeI and XhoI restriction enzymes and cloned into a pET21a expression vector. The pET21a expression vector into which the DNA of the fusion protein was cloned was named pET21a-6H-Ellasgen, and it was confirmed by electrophoresis and sequence analysis whether the DNA of the fusion protein was inserted. As a result, as shown in FIG. 1, an expression vector expressing the fusion protein of type I collagen α1 and elastin was prepared.

### Example 2. Expression of Fusion Protein

The expression vector comprising the fusion protein of type I collagen α1 and elastin prepared above was transformed into E. coli to confirm expression.

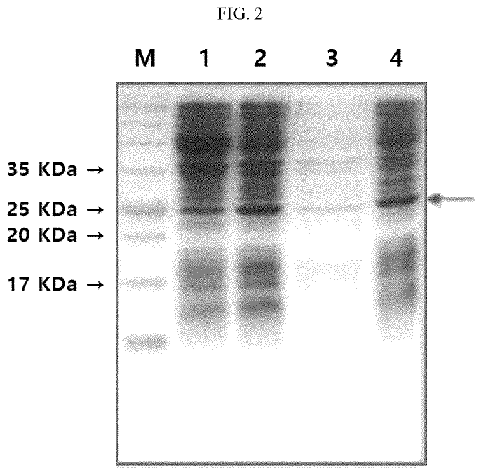

The pET21a-6H-Ellasgen expression vector prepared above was transformed into E. coli Rosetta 2 (DE3) by a conventional method, and the strain transformed with the expression vector was inoculated into 10 ml of LB liquid medium containing 50 μg/ml of ampicillin and shake-cultured at 37° C. for 18 hours. 300 μl of the cultured medium was inoculated into 10 ml of LB liquid medium and cultured until the OD₆₀₀ value reached 0.6. 1 mM of IPTG was added to the cultured strain, and expression of the fusion protein was induced by further culturing at 25° C. for 3 hours. After culturing, the cells were harvested by centrifugation, the harvested cells were suspended in 1 ml of 50 mM Tris-HCl (pH 8.0) buffer, and ultrasonicated (Sonifier 450, 3 kHz, 3 watts, 1 minute). The cell lysate was used to confirm the expression of the fusion protein by electrophoresis on polyacrylamide gel by a conventional method, and the results are shown in FIG. 2.

As shown in FIG. 2, it was confirmed that the fusion protein according to the present invention was expressed at a size of about 25 kDa.

### Example 3. Purification of Fusion Protein

The fusion protein of type I collagen α1 and elastin, whose expression was confirmed above, was purified by the following method, and the purified fusion protein was confirmed.

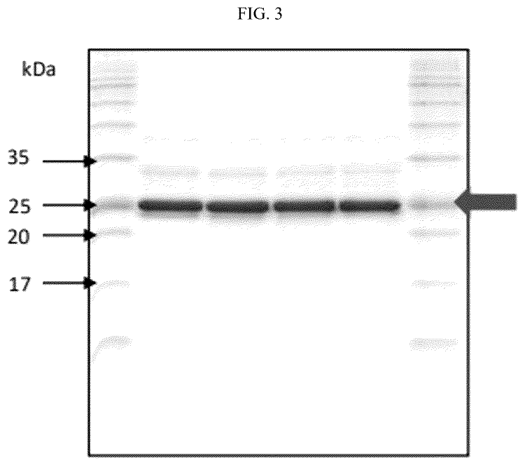

First, the E. coli Rosetta 2 (DE3) strain transformed with the pET21a-6H-Ellasgen expression vector was inoculated into 10 ml of LB liquid medium containing 50 μg/ml of ampicillin and shake cultured at 37° C. for 18 hours. The cultured broth was inoculated into 1 l of LB liquid medium, and expression of the fusion protein was induced under the same conditions as described in Example 2. The cultured medium was centrifuged to harvest the cells, the harvested cells were suspended in 50 mM Tris-HCl (pH 8.0) buffer, and ultrasonicated. The lysed cells were centrifuged to harvest the supernatant, and the fusion protein was purified from the harvested supernatant by a conventional method using Sepabeads resin brominated styrenic adsorbent (Sorvent Technologies, Inc., USA). The purified protein was confirmed by 15% SDS-PAGE electrophoresis, and the resulting photograph is shown in FIG. 3.

As shown in FIG. 3, the fusion protein according to the present invention was purified at a size of about 25 kDa.

### Experimental Example 1. Cytotoxicity

The cytotoxicity of the fusion protein of type I collagen α1 and elastin prepared above was confirmed by the MTT assay method as follows.

First, HDF cells (human dermal fibroblast, ATCC, USA), which are human primary skin cells, were prepared by subculturing in DMEM (Dulbecco's Modified Eagle's medium) culture medium containing 1% antibiotic-antimycotic and 10% FBS (fetal bovine serum) under conditions of 37° C. and 5% CO₂. The prepared cells were seeded into a 24-well plate at 5×10⁴ cells/well and cultured for 24 hours. Meanwhile, the fusion protein of type I collagen α1 and elastin was diluted in the culture medium to prepare 0, 2.5, 5, 10, 20, or 40%. Afterward, the cultured cells were treated with the fusion protein and cultured under the same conditions for 24 hours, and 0.05% MTT solution was added and further cultured for 4 hours. After culturing, the culture medium was removed, and 1 ml of DMSO (dimethyl sulfoxide) was added and mixed well. The absorbance was measured at a wavelength of 540 nm, and the cell viability was calculated from the measured absorbance value by a conventional method, and the results are shown in Table 1.

TABLE 1

| Protein Concentration (%) | Cell Viability (%) |
|---|---|
| 0 | 100.00 |
| 2.5 | 100.56 |
| 5 | 103.03 |
| 10 | 102.21 |
| 20 | 102.06 |
| 40 | 102.78 |

As shown in Table 1, the fusion protein according to the present invention did not show cytotoxicity.

### Experimental Example 2. Promotion of Cell Proliferation

The cell proliferation-promoting effect of the fusion protein of type I collagen α1 and elastin prepared above was confirmed by the following method.

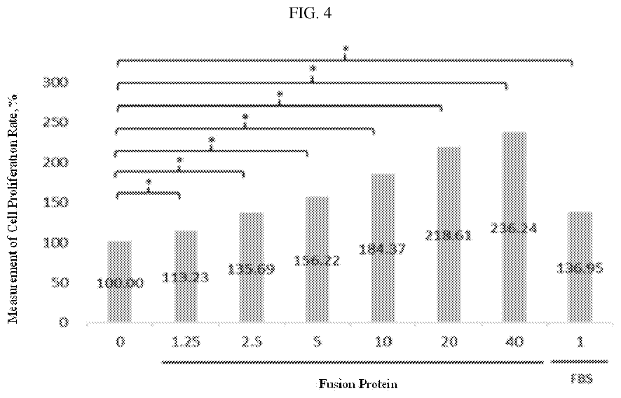

First, HDF cells were prepared by the same method and conditions as in Experimental Example 1, and samples of the fusion protein of type I collagen α1 and elastin were prepared by diluting to concentrations of 1.25, 2.5, 5, 10, 20, or 40%. The prepared cells were seeded into a 96-well plate at 1×10⁴ cells/well and cultured for 24 hours. The cultured cells were washed twice with PBS buffer and further cultured under the same conditions after treating the prepared samples. After 24 hours, the absorbance was measured at a wavelength of 450 nm using a BrdU cell proliferation ELISA kit. At this time, medium not containing FBS was used as a negative control, and FBS was used as a positive control. The cell proliferation rate was calculated from the measured absorbance value by a conventional method, and the results are shown in Table 2 and FIG. 4.

TABLE 2

| Protein Concentration (%) | Cell Proliferation Rate (%) |
|---|---|
| Negative Control | 100.00 |
| Positive Control | 136.95 |
| 1.25 | 113.23 |
| 2.5 | 135.69 |
| 5 | 156.22 |
| 10 | 184.37 |
| 20 | 218.61 |
| 40 | 236.24 |

As shown in Table 2 and FIG. 4, the fusion protein according to the present invention promoted cell proliferation in a treatment concentration-dependent manner.

### Experimental Example 3. Promotion of Collagen Production

The wrinkle improvement effect of the fusion protein of type I collagen α1 and elastin prepared above was confirmed by measuring the collagen production rate.

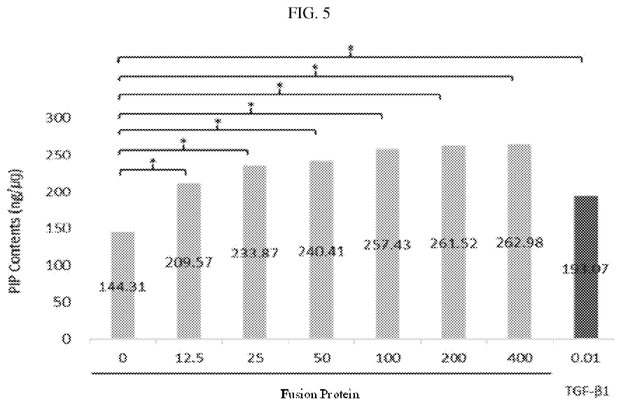

First, HDF cells were prepared by the same method and conditions as in Experimental Example 1, and the fusion protein of type I collagen α1 and elastin was prepared at concentrations of 12.5, 25, 50, 100, 200, or 400 μg/ml by confirming the concentration by a conventional method. The prepared cells were seeded into a 24-well plate at 5×10⁴ cells/well and cultured for 24 hours. The cultured cells were washed twice with PBS buffer and treated with the prepared samples, followed by further culturing under the same conditions. After 24 hours, the cells were harvested and centrifuged at 10,000 rpm for 10 minutes to obtain the supernatant. Using the obtained supernatant as a sample, absorbance was measured at a wavelength of 450 nm with a Procollagen Type I C-Peptide ELISA kit (PIP ELISA kit). At this time, FBS-free medium was used as a solvent control, and 0.01 μg/ml of TGF-β1 was used as a positive control. The results of confirming the collagen production amount from the measured absorbance value by a conventional method are shown in FIG. 5.

As shown in FIG. 5, the fusion protein according to the present invention promoted collagen production in a treatment concentration-dependent manner.

### Experimental Example 4. Inhibition of Collagenase (MMP-1) Activity

The wrinkle improvement effect of the fusion protein of type I collagen α1 and elastin prepared above was confirmed through the MMP-1 activity inhibition effect.

### 4-1. Intracellular Inhibitory Effect

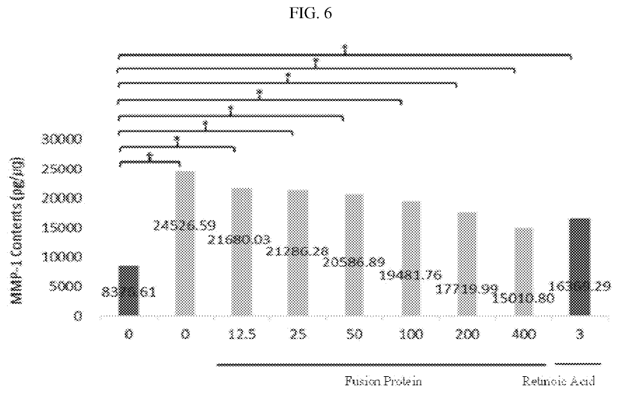

First, HDF cells were prepared by the same method and conditions as in Experimental Example 1, and the fusion protein of type I collagen α1 and elastin was prepared at concentrations of 12.5, 25, 50, 100, 200, or 400 μg/ml by confirming the concentration by a conventional method. The prepared cells were seeded into a 24-well plate at 5×10⁴ cells/well and cultured for 24 hours. After culturing, the medium was removed, PBS was added, and ultraviolet (UVB) rays of 10 mJ/cm² were irradiated. The cells irradiated with ultraviolet rays were treated with the prepared samples and further cultured under the same conditions. After 24 hours, the cells were harvested and centrifuged at 10,000 rpm for 10 minutes to obtain the supernatant. Using the obtained supernatant as a sample, absorbance was measured at a wavelength of 450 nm with an MMP-1 human ELISA kit. At this time, FBS-free medium was used as a solvent control, and 3 μg/ml of retinoic acid was used as a positive control. The results of calculating the collagenase activity from the measured absorbance value according to the following Equation 1 are shown in FIG. 6.

$$Collagenase activity ( \% ) = \frac{{OD}_{Test} - {OD}_{COlor}}{{OD}_{Control} - {OD}_{Color}} \times 100 \qquad [ Equation 1 ]$$

OD_{Test}: Absorbance of Sample Reaction Solution
OD_{Control}: Absorbance of the Solvent Control Reaction Solution
OD_{Color}: Absorbance of Sample and Solvent Control

As shown in FIG. 6, the fusion protein according to the present invention inhibited collagenase activity in a treatment concentration-dependent manner.

### 4-2. Inhibitory Effect by Substrate-Enzyme Reaction

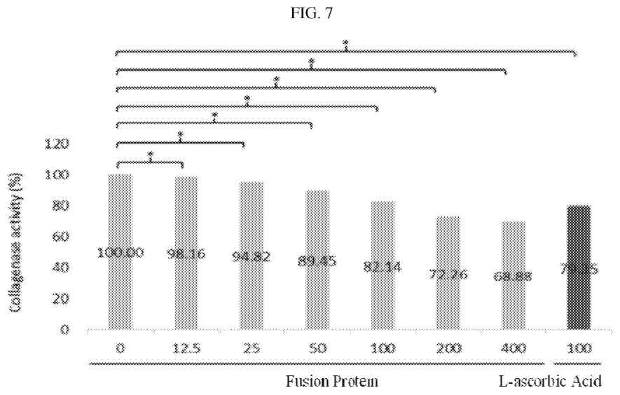

First, the fusion protein of type I collagen α1 and elastin was prepared at concentrations of 12.5, 25, 50, 100, 200, or 400 μg/ml by confirming the concentration by a conventional method. To the prepared fusion protein, 2 mg of azocoll, a substrate of collagenase, and 100 μl of collagenase (1 mg/ml) were added, and distilled water was added so that the total reaction volume became 1 ml. The reaction solution was stirred at 37° C. for 1 hour and centrifuged at 10,000 rpm for 10 minutes to obtain the supernatant. 200 μl of the obtained supernatant was dispensed into a 96-well plate, and absorbance was measured at a wavelength of 550 nm. At this time, 100 μg/ml of L-ascorbic acid was used as a positive control, and distilled water was used as a solvent control. The results of calculating the collagenase activity from the measured absorbance value according to Equation 1 are shown in FIG. 7.

As shown in FIG. 7, the fusion protein according to the present invention inhibited collagenase activity in a treatment concentration-dependent manner.

Therefore, from the above results, it was found that the fusion protein according to the present invention exhibits a wrinkle improvement effect by promoting collagen synthesis and simultaneously inhibiting collagenase activity.

1. A fusion protein comprising a fragment of type I collagen α1 and a fragment of elastin,
wherein the fusion protein is a polypeptide consisting of the amino acid sequence set forth in SEQ ID NO: 5.

2. A polynucleotide encoding the fusion protein according to claim 1.

3. An expression vector comprising the polynucleotide according to claim 2.

4. A transformant transformed with the expression vector of claim 3.

5. A method for preparing a fusion protein of a fragment of type I collagen α1 and a fragment of elastin, comprising the step of culturing the transformant of claim 4,
wherein the fusion protein is a polypeptide consisting of the amino acid sequence set forth in SEQ ID NO: 5.

6. A cosmetic composition for skin improvement comprising the fusion protein of claim 1 as an active ingredient.

7. The cosmetic composition for skin improvement according to claim 6, wherein the skin improvement is due to skin wrinkle improvement.


SEQUENCE LISTING (10 sequences)

SEQ ID NO: 1 - protein, 163 aa, synthetic construct
GPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAG

SEQ ID NO: 2 - DNA, 489 bp, synthetic construct
  source 1..489: mol_type = other DNA
ggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggc

SEQ ID NO: 3 - protein, 123 aa, synthetic construct
PGVGGVPGVGGVPGVGISPEAQAAAAAKAAKYGAAGAGVLGGLVPGAPGAVPGVPGTGGVPGVGTPAAAAAKAAAKAAQFGLVPGVGVAPGVGVAPGVGVAPGVGLAPGVGVAPGVGVAPGVG

SEQ ID NO: 4 - DNA, 369 bp, synthetic construct
  source 1..369: mol_type = other DNA
cctggtgtcggaggtgttcccggagtcggaggtgtcccgggagttggcatttcccccgaagctcaggcagcagctgccgccaaggctgccaagtacggtgctgcaggagcaggagtgctgggtgggctagtgccaggtgccccaggcgcagtcccaggtgtgccgggcacgggaggagtgccaggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggc

SEQ ID NO: 5 - protein, 286 aa, synthetic construct
GPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGPGVGGVPGVGGVPGVGISPEAQAAAAAKAAKYGAAGAGVLGGLVPGAPGAVPGVPGTGGVPGVGTPAAAAAKAAAKAAQFGLVPGVGVAPGVGVAPGVGVAPGVGLAPGVGVAPGVGVAPGVG

SEQ ID NO: 6 - DNA, 858 bp, synthetic construct
  source 1..858: mol_type = other DNA
ggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggccctggtgtcggaggtgttcccggagtcggaggtgtcccgggagttggcatttcccccgaagctcaggcagcagctgccgccaaggctgccaagtacggtgctgcaggagcaggagtgctgggtgggctagtgccaggtgccccaggcgcagtcccaggtgtgccgggcacgggaggagtgccaggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggc

SEQ ID NO: 7 - protein, 1158 aa, synthetic construct
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 8 - protein, 753 aa, synthetic construct
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGAAGAGVLGGLVPGAPGAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 9 - DNA, 44 bp, synthetic construct
  source 1..44: mol_type = other DNA
ggagatatacatatgcatcaccatcatcaccatggtcctcctgg

SEQ ID NO: 10 - DNA, 30 bp, synthetic construct
  source 1..30: mol_type = other DNA
gtggtggtgctcgagttagccaacgccagg